(19) United States
(12) Patent Application Publication
(10) Pub. No.: US 2025/0099778 A1
(43) Pub. Date: Mar. 27, 2025

(54) PHOTON BEAUTY INSTRUMENT

(71) Applicant: SHENZHEN JVK MEDICAL INSTRUMENTS CO., LTD., Shenzhen City, Guangdong Province (CN)

(72) Inventors: YING ZHOU, Shenzhen City, Guangdong Province (CN); BING LI, Shenzhen City, Guangdong Province (CN)

(21) Appl. No.: 18/730,756

(22) PCT Filed: Oct. 9, 2022
(86) PCT No.: PCT/CN2022/123945

§ 371 Date: Jul. 19, 2024

(30) Foreign Application Priority Data
Jan. 19, 2022 (CN) 202210061948.2

(51) Int. Cl. A61N 5/06

(52) U.S. Cl. CPC A61N 5/0616; A61N 2005/0658; A61N 2005/0667

(57) ABSTRACT

The present application relates to a photon beauty instrument. The photon beauty instrument includes a shell, in which a light source assembly, a power source unit and a control circuit board are provided, the light source assembly and the power source unit are electrically connected to the control circuit board. A light output channel and a light outlet are arranged inside the head of the photon beauty instrument, and light from the light source assembly are transmitted via the light output channel and are emitted from the light outlet. A filtering assembly, in which filters can be switched, includes one or more filters, or comprises a filtering film which has a plurality of filtering areas, and each filter or each filtering area of the filtering film can be switched and move to the light output channel for filtering light.

### FIELD OF THE INVENTION

The present invention relates to the field of beauty equipment, and more particularly, to a photon beauty instrument.

### DESCRIPTION OF BACKGROUND ART

Photon beauty devices use pulsed light or laser or other light sources to implement beauty functions, which are called photon beauty devices. The light source assembly inside the photon beauty device generates light waves, which are emitted from the light outlet window of a working head of the photon beauty device to perform beauty treatments on the skin surface in contact (or non-direct contact) with an end face of the working head, such as hair removal, skin rejuvenation, spot removal, anti-inflammation, blood vessel softening, wrinkle removal, skin redness removal, acne treatment, vascular lesions treatment, pigmented lesions treatment, etc. Some portable or handheld photon beauty devices currently on the market, for obtaining desired light waves, need to carry multiple optical filters or multiple accessory heads, which must be inserted and replaced during use. Therefore, they are inconvenient to use, carry, and store.

### SUMMARY OF THE INVENTION

### Technical Problem

The technical problem to be solved by the invention is to provide a photon beauty instrument that solves the problems of the existing photon beauty instrument with replaceable optical filters being inconvenient to use, carry, and store.

### Technical Solutions

In order to solve the above technical problems, the present invention adopts the following technical solutions:

A photon beauty instrument, comprises a housing, and a light source assembly, a power source unit and a control circuit board arranged in the housing; the light source assembly, the power source unit and the control circuit board are electrically connected; a head of the photon beauty instrument defines a light outlet channel and a light outlet therein; light from the light source assembly is transmitted through the light outlet channel and emitted from the light outlet; the head of the photon beauty instrument is provided with an optical filter assembly with switchable optical filters therein; the optical filter assembly comprises one or more optical filters, or an optical filter film having multiple optical filter areas; and each optical filter or each optical filter area of the optical filter film is movable and switchable to enter the light outlet channel for filtering light transmitted through the light outlet channel.

Further, multiple optical filters are connected in series by a connecting mechanism or spliced together to form linked optical filters; the linked optical filters are transported to the light outlet channel using a transmission mechanism or an adjustment mechanism for filtering light in the light outlet channel; or, one or more optical filters are each movably arranged in side walls of the light outlet channel, and are moved or rotated into the light outlet channel for filtering light transmitted through the light outlet channel.

Further, a holder is provided in the head of the photon beauty instrument, and the holder defines the light outlet channel; a side wall of the light outlet channel is provided with a slot or a rotating mechanism; the one or more optical filters or the optical filter film are moved into the light outlet channel through the slot for filtering light transmitted through the light outlet channel, or the one or more optical filters are rotated by the rotating mechanism into the light outlet channel for filtering light transmitted through the light outlet channel.

In some embodiments, the slot is disposed in the holder for storing the optical filters or the optical filter film; the optical filters or the optical filter film are movable forward and backward and transported along the slot into the light outlet channel for filtering light.

In some embodiments, the slot is arranged in the holder along a length direction of the holder and penetrate opposite sides of the holder; the optical filters are stored in the slot along the length direction and movable back and forth along the slot in the light outlet channel.

In some embodiments, the optical filter is divided into two sub-optical filters, which are movable or rotatable to enter the light output channel and be spliced into one entire optical filter.

In some embodiments, a storage cavity is formed outside the side wall of the light outlet channel, and is connected to the light outlet channel through the slot defined through the side wall of the light outlet channel; the optical filters or the optical filter film pass through the slot, are movable back and forth between the storage cavity and the light outlet channel, whereby being stored in the storage cavity or crossing the light outlet channel for filtering light.

In some embodiments, the optical filters or optical filter film are arc-shaped or planar; positions of the optical filters or optical filter film in the optical filter assembly are manually switchable using a manual turning member or a lever or a connecting rod; or, positions of the optical filters or optical filter film are automatically adjustable by means of a motor driving a gear transmission, or a belt transmission, or a chain transmission, or a screw rod.

In some embodiments, the optical filter assembly is arranged in the head of the photon beauty instrument, the holder is a lamp holder, the light source assembly is installed in one side of the lamp holder; the light source assembly includes a lamp and a reflector, and an opening side of the reflector form a light outlet window, a fixed optical filter covers the light outlet window to seal the opening side of the reflector; a heat sink is provided outside the reflector; a fan is installed in the head of the beauty instrument, and a housing of the head defines air inlets and air outlets, whereby air cooling and heat dissipation for the light source assembly is available.

In some embodiments, the optical filter assembly is installed in an accessory head, the accessory head as an external component is connected to the head of the photon beauty instrument; a light outlet window is provided in a center of a top surface of the accessory head as a light entrance, which is connected with the light outlet of the head of the photon beauty instrument; a fixed bracket includes a pair of parallel side walls with a space therebetween forming a light outlet channel and a light outlet, and a space between the parallel side walls and an outer shell of the accessory head forms a storage cavity for the optical filters being stored therein or moving in or out; through slots are defined in the parallel side walls respectively, connects the light outlet channel of the accessory head to the storage cavity, whereby the optical filters are slidable into or out of the light outlet channel.

### Advantages

The photon beauty instrument of the present invention is provided with an optical filter assembly inside or outside the head of the beauty instrument for replacing optical filters; the optical filter assembly includes one or more optical filters or includes an optical filter film having one or more optical filter areas, and it is easier to replace optical filters, operate, carry and store.

The present invention will be described in further detail below in conjunction with the accompanying drawings.

### BRIEF DESCRIPTION OF DRAWINGS

FIG. 1 illustrates an exploded view of a photon beauty instrument in accordance with a first embodiment of the present invention;

FIG. 2 illustrates a cross-sectional view of the photon beauty instrument along an axis direction in accordance with the first embodiment of the present invention;

FIG. 3 illustrates another cross-sectional view of the photon beauty instrument along the axis direction in accordance with the first embodiment of the present invention;

FIG. 4 illustrates an internal structure of a working head of the photon beauty instrument in accordance with the first embodiment of the present invention; where (a)-(c) represent optical filters at different states of in the working head;

FIG. 5 illustrates plan views of the internal structure of the working head of the photon beauty instrument in accordance with the first embodiment of the invention, where (a)-(c) represent the optical filters at different states of in the working head;

FIG. 6 illustrates perspective views of the internal structure of the working head of the photon beauty instrument in accordance with the first embodiment of the invention, where (a)-(c) represent the optical filters at different states of in the working head;

FIG. 7 illustrates the optical filter assembly of the photon beauty instrument in accordance with the first embodiment of the invention, where (a)-(b) are different exploded views;

FIG. 8 illustrates the optical filter assembly of the photon beauty instrument in

accordance with the first embodiment of the invention, where (a)-(c) represent the optical filters at different states;

FIG. 9 illustrates a perspective view of another embodiment of the optical filter assembly of the photon beauty instrument in accordance with the first embodiment of the invention;

FIG. 10 illustrates the photon beauty instrument in accordance with a second embodiment of the present invention, where (a) is a perspective view, and (b) is a cross-sectional view along an axis direction;

FIG. 11 illustrates the internal structure of the working head of the photon beauty instrument in accordance with the second embodiment of the present invention, where (a)-(c) represent the optical filters at different states;

FIGS. 12 to 14 illustrate the internal structure of the working head of the photon beauty instrument in accordance with a third embodiment of the present invention; wherein 12(a), 13(a), and 14(a) show the states of the movable optical filter when there is no fixed optical filter; 12(b), 13(b), and 14(b) show the states of the movable optical filter when there is a fixed optical filter;

FIGS. 15 to 19 illustrate various optical filter assembly of the photon beauty instrument in accordance with the third embodiment of the present invention;

FIG. 20 illustrates a perspective view of the optical filter of the photon beauty instrument in accordance with the third embodiment of the present invention;

FIG. 21 illustrates a perspective view of the photon beauty instrument in accordance with a fourth embodiment of the present invention;

FIG. 22 illustrates a partial cross-sectional view of the photon beauty instrument in accordance with the fourth embodiment of the present invention;

FIG. 23 illustrates the internal structure of the working head of the photon beauty instrument in accordance with the fourth embodiment of the present invention, where (a)-(c) represent the states of the optical filters;

FIG. 24 illustrates the internal structure of the working head of the photon beauty instrument in accordance with a fifth embodiment of the present invention;

FIG. 25 illustrates a cross-sectional view of the internal structure of the working head of the photon beauty instrument in accordance with the fifth embodiment of the present invention;

FIG. 26 illustrates the optical filter of the photon beauty instrument in accordance with the fifth embodiment of the present invention, where (a)-(c) represent the states of the optical filter;

FIG. 27 illustrates a partial cross-sectional view of the photon beauty instrument in accordance with a sixth embodiment of the present invention;

FIG. 28 illustrates a perspective view of the photon beauty instrument in accordance with the sixth embodiment of the present invention;

FIG. 29 illustrates the internal structure of the working head of the photon beauty instrument in accordance with the sixth embodiment of the present invention, where (a)-(c) represent the switching state of the optical filters;

FIG. 30 illustrates the internal structure of the working head of the photon beauty instrument in accordance with a seventh embodiment of the present invention, where (a)-(c) represent the switching state of the optical filters;

FIG. 31 illustrates a partial cross-sectional view of the photon beauty instrument in accordance with an eighth embodiment of the present invention;

FIG. 32 illustrates a perspective view of the photon beauty instrument in accordance with the eighth embodiment of the present invention;

FIG. 33 illustrates the optical filter of the photon beauty instrument in accordance with the eighth embodiment of the present invention, where (a)-(c) represent the switching state of the optical filters;

FIG. 34 illustrates the optical filter assembly of the photon beauty instrument in accordance with the eighth embodiment of the present invention, where (a)-(b) represent the switching state of the optical filters;

FIG. 35 illustrates a partial structural of the optical filter assembly of the photon beauty instrument in accordance with the eighth embodiment of the present invention;

FIG. 36 illustrates the optical filter of the photon beauty instrument in accordance with the eighth embodiment of the present invention, where (a)-(b) represent the front and back of the optical filter respectively;

FIG. 37 illustrates the optical filter assembly of the photon beauty instrument in accordance with a ninth embodiment of the present invention, where (a)-(c) represent the switching state of the optical filters;

FIG. 38 illustrates a perspective view of an external accessory head of the photon beauty instrument of the present invention;

FIG. 39 illustrates cross-sectional views of the external accessory head of a first embodiment of the photon beauty instrument of the present invention, where (a)-(c) represent the switching state of the optical filters;

FIG. 40 illustrates a cross-sectional view of a second embodiment of the external accessory head of the photon beauty instrument of the present invention;

FIG. 41 illustrates a partial enlarged view of FIG. 40;

FIG. 42-44 illustrate cross-sectional views of the optical filter switching state in a third embodiment of the external accessory head of the photon beauty instrument of the present invention;

FIG. 45-47 illustrates cross-sectional views of the optical filter switching state in a fourth embodiment of the external accessory head of the photon beauty instrument of the present invention; and

FIG. 48 illustrates an exploded view of the photon beauty instrument in accordance with a tenth embodiment of the present invention.

### DETAILED DESCRIPTION OF THE INVENTION

It should be noted that, without conflict, the embodiments and features in the embodiments in the present invention can be combined with each other. The present invention will be further described in detail below with reference to the drawings and specific embodiments.

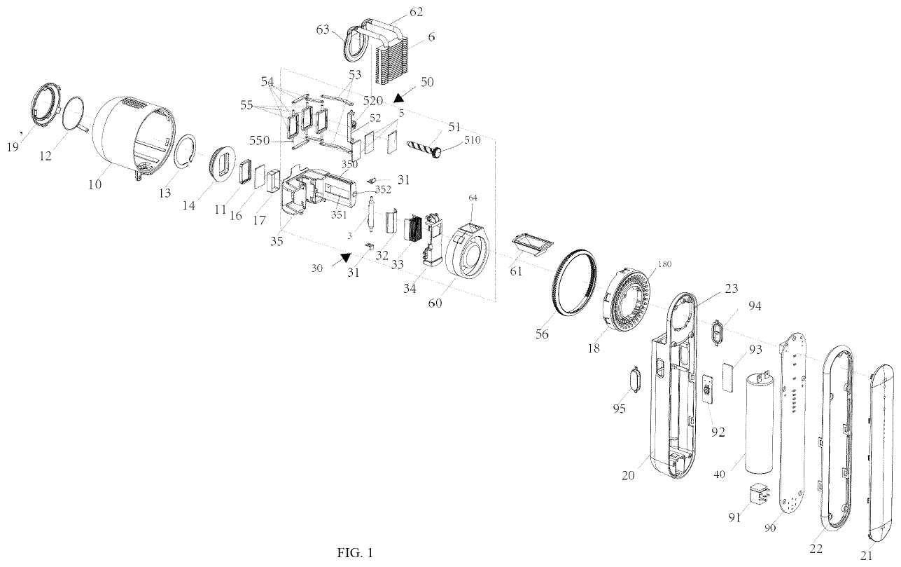

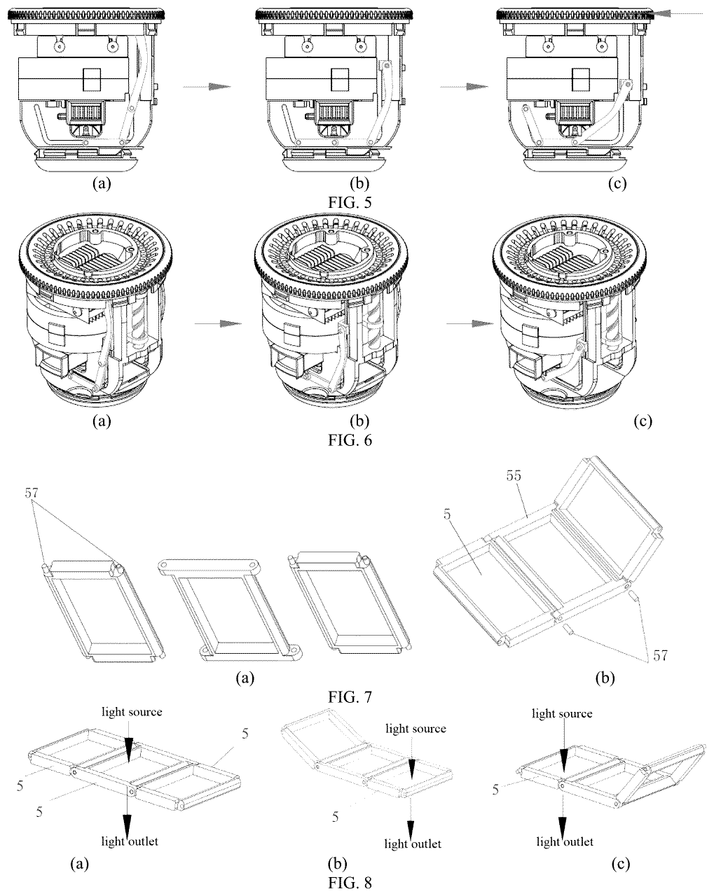

Please refer to FIGS. 1-48, a photonic beauty instrument provided in accordance with embodiments of the present invention, has a built-in or external optical filter assembly 50 with switchable optical filters. The optical filter assembly 50 includes one or more optical filters 5 or includes an optical filter film 5 with one or more optical filter areas; each optical filter or optical filter area of the optical filter film can be moved to a light output channel to perform filtering on the emitted light waves in the light output channel. The switchable optical filters can be moved automatically or manually for filtering the light waves generated by the light source assembly 30 in the beauty instrument to obtain the emitted lights with desired photon bands, so as to process the skin and obtain corresponding beauty effects.

When the optical filter assembly is built-in, the photon beauty instrument of the present invention is provided with an optical filter assembly 50 in the working head. The optical filter assembly 50 includes a plurality of optical filters. A manual adjustment mechanism is provided on the housing of the beauty instrument, or an electric adjustment mechanism is provided inside the beauty instrument to drive and change the position of the optical filter, thereby switching the optical filter behind the light outlet of the beauty instrument to optical filter light. The filtered light waves are emitted from the light outlet of the working head.

For the external optical filter assembly, generally the accessory head can be connected to the working head of the beauty instrument. The accessory head has multiple optical filters built into it. A manual adjustment mechanism is provided on a housing of the accessory head, or an electric adjustment mechanism is provided inside the beauty instrument and is connected to the optical filters of the accessory head, thereby switching the optical filter behind the light outlet of the accessory head to optical filter light. The filtered light waves are emitted from the light outlet of the accessory head.

A main body of the photon beauty instrument can be a hair removal instrument, and the optical filter assembly 50 is arranged in the working head of the hair removal instrument, or the accessory head is connected to the working head of the hair removal instrument, so as to manually or electrically switch of the optical filters.

The photon beauty instrument of the present invention includes a head (i.e. working head) 1 and a body 2. A body housing and a head housing are integral structure or are fixedly or removably assembled to form a housing of the photon beauty instrument. A light source assembly 30, a power source unit 40 and a control circuit board 90 are provided inside the housing. The power source unit 40 supplies power to the light source assembly 30. The control circuit board 90 controls the light source assembly 30 to generate light waves. In one embodiment, the light source assembly 30 generates pulsed light waves. A front end of the working head of the photon beauty instrument forms a light outlet. The housing of the working head, that is, the front housing 10, is provided with air inlets 101 and air outlets 102. The air inlets 101 and the air outlets 102 can be one or more sets of through holes defined in the housing, or they can be gaps between shells, and are used for air-cooling and heat dissipation of the light source assembly. The air inlets and air outlets can be set corresponding to the position of the light source assembly inside the beauty instrument.

In some embodiments, an optical filter assembly 50 is installed in the working head of the photon beauty instrument. During operation, the light waves generated by the light source assembly 30 are filtered by the optical filter assembly 50 and then transmitted to the light outlet for beauty or therapeutic effects. The light waves filtered by the optical filter assembly 50 have different photon wavebands so as to obtain different effects on the skin. For example, photons with a wavelength of 480 nm have the function of sterilization and disinfection, photons with a wavelength of 530 nm can rejuvenate skin and lighten spots, photons with a wavelength of 600 nm can soften blood vessels, and photons with a wavelength of 640 nm can remove hair. According to effects of photons, it is necessary to use photons of different wavelengths for different skin problems. Optical filters of different wavelengths are provided for switching during use.

An optical filter assembly 50 is provided in the working head 1 of the photon beauty instrument. The optical filter assembly 50 includes a plurality of optical filters 5. After the optical filter assembly 50 is installed in the working head 1, different optical filters 5 can be switched manually or automatically. There is no need to carry multiple accessories or multiple optical filters separately. The structure of the optical filter assembly is simple and the operation is convenient.

Referring to FIGS. 1-9, in a first embodiment of the photon beauty instrument, a light source assembly 30 is installed in the working head, and the optical filter assembly 50 is installed between the light source assembly 30 and the light outlet. The control circuit board 90 is electrically connected to the light source assembly 30 and the power source unit 40, and controls the light source assembly to generate light waves. The power source unit 40 is used to power the light source assembly 30. A front end of the working head 1 of the photon beauty instrument is provided with a light outlet, and a front end surface is used as a working surface that can be in contact with the skin for beauty treatment. The control circuit board 9 controls the light source assembly 30 to generate pulsed light such as Intense pulsed light (IPL). The pulsed light is filtered by the filtering device 50 and then emitted for beautifying the skin.

Specifically, the housing of the body 2 includes a lower cover 20 and a display cover 21 that is fastened with the lower cover. A decorative ring cover 22 can also be provided between the lower cover 20 and the display cover 21. The covers are fastened and define a cavity therein, and the control circuit board 90 and the power source unit 40 are installed in the cavity. The power unit 40 may be a rechargeable battery, a disposable battery, a capacitor battery, an external power module, or other power modules in the prior art. The control circuit board 90 is provided with a power socket 91, such as a DC power socket, for external power connection or internal power connection. The body 2 is provided with buttons, such as a power-on button 95, a lighting button 94, etc. The body 2 is provided with a lighting PCB 93 and a key circuit board 92 therein, which are electrically connected to the control circuit board 90 respectively.

One end of the lower cover 20 forms a mounting plate 23 for installing and connecting the working head 1. The mounting plate 23 may be a part of the lower cover 20, with a central hole for connection between the light source assembly 30 and the control circuit board 90 in the body 2. In this embodiment, the mounting plate 23 is annular and is connected to the working head 1 through fasteners. The working head 1 is connected to the body 2, forming an L shape as a whole, and the body 2 serves as the handheld.

The working head housing includes a front housing 10 with a cylindrical structure, and has an internal cavity equipped with a light source assembly 30, an optical filter assembly 50 and a heat dissipation assembly. The heat dissipation assembly includes a fan 60. The air inlets 101 and the air outlets 102 can be defined in the front housing 10, corresponding to the positions of the fan 60, and are mainly used for heat dissipation of the light source assembly 30. The fan 60 is installed between the light source assembly 30 and the mounting plate 23 of the body 2. Cold air entering through the air inlets 102 is directed to the light source assembly 30 and then enters the fan through the air inlet on one side of the fan 60. An air outlet 64 is provided in a side of a fan spiral housing and is in air communication with the air outlets 102 in the front housing 10. The air outlet 64 of the fan spiral housing is connected to an air guide 61. The air guide 61 guides hot air to the air outlets 102 and discharges hot air to the outside of the body 2. The air guide 61 is conducive to improving heat dissipation efficiency. The shape of the rear end of the front housing 10 is adapted to the mounting plate 23. In this embodiment, the front housing 10 is cylindrical with a reduced diameter at a front end, and the front-end opening forms a light outlet. An annular air inlet cover 18 is further provided between the front housing 10 and the mounting plate 23. A rear end of the front housing 10 and the air inlet cover 18 are installed to the installation plate 23, and are clamped and connected by fasteners. Through holes 180 is defined along the circumference of the annular air inlet cover 18 as air inlets. The front end of the front housing 10 may be further equipped with an annular light panel 13, a skin induction coil 12 (or sensor), and a front decorative ring 19′. The annular light panel 13 and the skin induction coil 12 (or sensor) are electrically connected to the control circuit board 90.

The light source assembly 30 includes a lamp 3, which is electrically connected to the control circuit board 90, and the electrodes 31 are arranged at both ends of the lamp. A reflector 32 is provided outside the lamp 3. A lamp holder 35 is provided in the working head 1, and the light source assembly 30 is installed to the lamp holder 35. A light outlet window is provided at a front end of the lamp holder 35 according to the light emission direction of the lamp 3. A reflector mask 17 is installed on the light outlet window (defining a light output channel therein for transmitting light to the light outlet) and a white glass 16 installed at the front end of the reflector mask 17. The transparent glass is fixed at the front end of the reflector mask 17 by a sealing ring 11. The working head 1 is further provided with an air guide sleeve 34, and an air guide sleeve 34 is used to install the light source assembly to the lamp holder 35. The air guide sleeve 34 is provided with an air guide channel, which is in air communication with the air inlet of the fan 60 to improve the heat dissipation efficiency of the reflector. It can be understood that the white glass 16 can be replaced by a fixed optical filter, or transparent glass, or the white glass is not necessary accordingly.

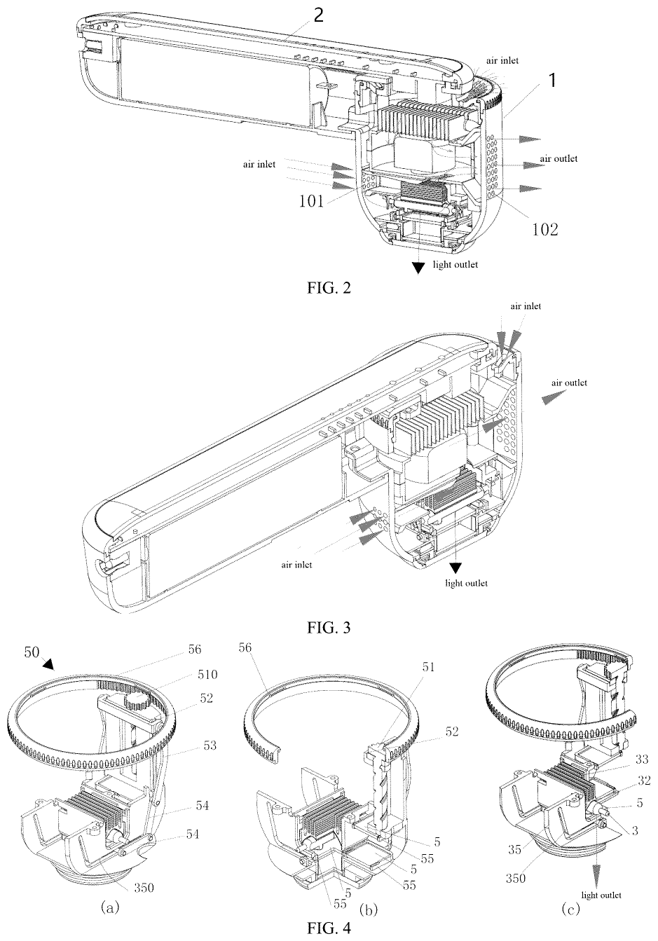

The light source assembly 30 is provided with a light source heat sink 33. The light source heat sink 33 may be a fin heat radiator, heat dissipation sheets, a heat conductive plate, a heat pipe, or the like. In a specific example, a fin radiator or heat sink is provided outside the lamp reflector 32. The heat sink 33 includes a thermal conductive base that is installed closely with the reflector. The fin radiator or heat sink is disposed on the surface of the thermal conductive base to conduct heat. The surface of the base fits the reflector and is consistent with a shape of the reflector, so as to quickly conduct heat from the reflector to the heat sink 33 for heat dissipation. The light source heat sink 33 is installed in the air guide sleeve 34. The air inlets 101 provided in the front housing 10 corresponds to the light source heat sink 33. Cold air enters the surface spaces of the light source heat sink 33 in the working head from the air inlets 101, taking away the heat for heat dissipation. The hot air is guided by the air guide sleeve 34, enters the fan through the fan air inlet, and is discharged from the fan air outlet 64 to the air outlets 102 of the front housing 10. The air inlets 101, air passage on the surface of the light source heat sink 33, the air passage of the fan 60, and the air outlets 102 are in air communication to form an air-cooling passage for the light source assembly, referring to the air-flow direction marked by the arrow in FIGS. 2-3.

As an embodiment, a piece of thermoelectric cooling is provided in the working head 1, and a cold side of the thermoelectric cooling is used to contact the skin to form an ice-cooling effect, or the cold side of the thermoelectric cooling is used to cool an end surface of the light outlet that is in contact with the skin, forming an ice-cooling effect on the contact skin surface. In one embodiment, the thermoelectric cooling 14 is installed outside the light outlet of the light source assembly, that is, installed in front of the light outlet window at the front end of the lamp holder 35, is adapted to the light outlet window and has a light-transmitting area. The light waves generated by the light source pass the light-transmitting area of the thermoelectric cooling. When working, a front-end surface of the thermoelectric cooling 14 can be used to contact the skin. The light-transmitting area of the thermoelectric cooling 14 is adapted to the light outlet window at the front end of the white glass 16 or the light outlet window of the lamp holder 35, and is covered on the white glass 16 or covered on the light outlet window of the lamp holder 35 so as to sealing the light output channel.

The working head 1 is also provided with a heat dissipation assembly for the thermoelectric cooling, including a heat sink 6 for the thermoelectric cooling, a heat pipe 62, and a heat conducting member 63 set on the heat pipe. The heat sink 6 is installed on the other side of the fan 60 and faces the light source heat sink 33. The heat sink 6 is arranged corresponds to the annular air inlet cover 18. The circumference of the annular air inlet cover 18 is provided with through holes 180 as air inlets. Cold air enters the working head from the through holes 180 and flow to the air space between the fins of the heat sink. Hot air enters the fan from the fan air inlet at the other side of the fan, and is discharged by the fan to the air outlets 102 of the front housing 10. The air path connecting between the through holes 180, the air space of the heat sink 6, the fan air inlet at the other side of the fan 60, and an air passage inside the fan, and the air outlets 102 are connected to form a cooling air passage for the thermoelectric cooling, referring to FIGS. 2-3, an air-flow direction marked by the arrows. The heat-conducting member 63 is connected with the thermoelectric cooling to cool the thermoelectric cooling. Specifically, the heat-conducting member is installed in close contact with a hot side of the thermoelectric cooling for rapid heat transfer. The heat conducting member 63 and the heat sink 6 are connected to the heat pipe 62. In this embodiment, the fan has air inlets at opposite sides, one air inlet at one side is used for the air inlet of the air-cooling passage for the light source assembly, and faces the light source heat sink 33/air guide sleeve; while the other air inlet at the other side of the fan is used for the air inlet of the air-cooling passage for the thermoelectric cooling, and faces the heat sink 6.

In other embodiments, the through holes 180 may also be a gap between the rear end of the front housing and the periphery of the mounting plate 23 of the body 2. The beauty instrument housing can be provided with multiple air inlets and outlets.

In the first embodiment, the optical filter assembly 50 is installed in the working head 1 and includes a plurality of optical filters 5 (three optical filters as shown in the figures). Multiple optical filters 5 are connected in a chain to form linked optical filters. Push one side or one end of the linked optical filters, so as to push different optical filter to the light outlet/light channel of the working head. Specifically, each optical filter 5 is fixed by an optical filter holder 55, and the optical filter holders 55 are connected together through rotating shafts, hinges or connecting rods.

As an embodiment, referring to FIGS. 1-6 and 9, the optical filter 5 is installed in the optical filter holder 55. Each optical filter holder 55 is provided with protruding shafts 550 at two ends or both sides, and a connecting rod 54 is movably connected to the protruding shafts 550 on either side of the two optical filter holders respectively, thereby movably connecting the optical filters. The connecting rod 54 is provided with a corresponding shaft hole, and the protruding shaft 550 is rotatably inserted in the shaft hole. In this embodiment, both ends of each connecting rod 54 are respectively provided with a shaft hole, which is sleeved with the convex shaft 550 on one side of each optical filter holder for rotation. A pair of connecting rods movably connected to the protruding shafts 550 on both sides of the filter holder 55 of the optical filter 5 at the end serves as first connecting rods 53. The first connecting rods 53 may be longer than other connecting rods as desired, and may be curved rods. The ends of the two first connecting rods 53 are fixedly connected by a slider 52, and the slider 52 is provided with an internally threaded shaft hole 520. In this embodiment, the optical filter assembly 50 also includes a screw rod 51. The screw rod 51 is inserted into the internally threaded shaft hole 520 of the slider 52. The threads cooperate to convert a rotational motion of the screw rod 51 into a linear motion of the slider 52. Refer to FIG. 2-6 and the direction shown in FIG. 9, the screw rod 51 can be arranged in the direction parallel to an axis of the working head 1, or arranged along an inner wall of the front housing 10 in the direction of the light outlet, so that the slider 52 can move back and forth in the direction of the light outlet. The reciprocating movement of the slider 52 can drive the first connecting rods 53 to move together, that is, drives the optical filters 5 connected by the connecting rods 53 and 54 to move together, and then drives the corresponding optical filters moving together forward or backward toward the light outlet, thereby move the corresponding optical filter to the light outlet/light outlet channel or away from the light outlet/light outlet channel to switch different optical filters 5. The lamp holder is L-shaped. A front section of the L-shaped lamp holder 35 is installed with the lamp 3 and is provided with a light outlet window, and a rear section of the L-shaped lamp holder 35 (along a direction parallel to the light emission direction/axis) is equipped with the slider 52 and a screw rod 51. The L-shaped lamp holder 35 is provided with slots 350 through both sides. The slot 350 is provided along a circumferential direction of the L-shaped lamp holder 35 for accommodating multiple linked optical filters 5 and guiding the optical filters 5 to move along the slot. The protruding shafts 550 on both sides of the optical filter holder 55 are respectively insert through the slots 350 and extend outward. Each pair of connecting rods 53 and 54 are rotationally connected to the corresponding protruding shafts 550 and are respectively located besides the slot 350. Preferably, a cross section of the slot is also L-shaped. The slot 350 is provided inside the lamp holder 35 and is used as a supporting track for moving and a storage space for multiple connected optical filters, and there is no need to set up a separate space or carry an additional optical filter 5.

The slider 52 is slidably disposed at the rear section (along a direction parallel to the light emission direction/axis) of the L-shaped lamp holder 35. The slider 52 is a crossbar with protruding shafts at both ends, and the protruding shafts are rotatably connected to the shaft holes at the end of the first connecting rods. A length of the slider 52 is adapted to a width of the rear section of the lamp holder 35. The entire slider 52 is slidably sleeved in the width of the rear section of the L-shaped lamp holder 35. The protruding shafts at both ends of the slider 52 extend outward and connect with the shaft hole at the ends of the first connecting rods. In this embodiment, the slider 52 and the connecting rods 53 and 54 form a linkage structure, they slide back and forth together toward the light outlet/light outlet channel and slide back and forth together along the rear section of the L-shaped lamp holder 35.

The rear section of the L-shaped lamp holder is a hollow structure, with a hollow space 351 formed in its center, and shaft holes 352 are provided in front and rear walls of the hollow space 351. The slider 52 is provided with an internally threaded shaft hole 520, which protrudes inward and is located in the hollow space 351. The screw rod 51 rotatably passes through the shaft holes 352 in the front and rear walls of the hollow space 351, and the screw rod 51 passes through the internally threaded shaft hole 520 of the slider 52 in the hollow space 351 and is threadedly engaged with each other so as to drive the slider 52 to move between the front and rear walls of the hollow space 351. The front and rear walls of the hollow space 351 serve as movement limits for the internally threaded shaft hole 520.

The rotation of the screw rod 51 can be implemented manually or electrically. A gear as an end cover 510 is provided at the end of the screw rod 51. The optical filter assembly 50 may also include an adjustment ring 56 that is rotatably mounted in the working head 1. In a specific example, the adjustment ring 56 is disposed at a rear annular end of the front housing 10 and can be rotated. The adjusting ring 56 can be set to be rotatable within a certain arc length (or a certain angle). The adjusting ring 56 is provided with internal teeth, which mesh with the external teeth of the gear end cover 510. Rotate the adjusting ring 56 to drive the gear end cover 510 (and the screw rod 51) to rotate. The rotatable arc length and the arc length provided with the internal gear teeth correspond to a circumference of the gear end cover 510. The adjustable ring 56 can be rotated forward and reverse to drive the gear end cover 510 (and the screw rod 51) to rotate forward and reverse, then drive the slider 52 to take optical filters 5 to move back and forth by the connecting rods, thereby switching different optical filter 5 to the light outlet window/light output channel at the front end of the lamp holder 35.

In other embodiments, a motor can also be provided in the working head and a gear is sleeved on the motor output shaft, and the gear is meshed with the gear end cover 510 provided at the end of the screw rod 51 to electrically move the optical filters. The motor is electrically connected to the control circuit board 90.

As an alternative embodiment, the optical filter 5 is fixed in the optical filter holder 55, two optical filter holders 55 are connected by a pivot or a hinge, forming a connection like a watch strap. Referring to FIGS. 7-8, there are shaft holes at both ends of the optical filter holder. A short shaft or short needle 57 is inserted into a pair of shaft holes of two adjacent optical filter holders to form a pivot connection, and each optical filter (optical filter holder) is connected in series to form linked optical filters. The two ends of the optical filter holder 55 of the first optical filter 5 can be driven by the screw rod 51 through the first connecting rods 53 and the slider 52 to move the linked optical filters to switch the optical filter located in the light outlet window/light output channel.

In other embodiments, other manual mechanisms can also be provided on the working head to connect the optical filters, and manually move the positions of the optical filters, so as to select and switch the optical filter at the light window.

The arrows in FIGS. 2-3 show the air-flow direction or the light emission direction, and the arrows in FIG. 4(c) and FIG. 8 show the light emission direction. The reciprocating movement direction of the slider 51 and the connecting rod and the series-connected optical filters driven by the slider are shown by the arrows in FIG. 9, and their movement paths can also be set according to the actual appearance of the instrument. FIGS. 4-6 show the switching states of series-connected optical filter.

In other embodiments, the slot 350 can also be defined between the lamp holder 35 and the inner wall of the front housing 10, as a receiving space for the optical filters, and used for the optical filter 5 to move and switching the optical filter at the light outlet.

Referring to FIGS. 10-11, the photon beauty instrument in a second embodiment is provided. FIG. 10(b) shows a simplified internal structure of the photon beauty instrument. The whole instrument is L-shaped, and the working head housing and the body housing can be of an integrated shell. In this embodiment, the optical filter assembly 50 includes multiple layers of optical filters 5, which are arranged in parallel and spaced apart in a pull-out type (like a drawer).

The selected optical filter is pushed forward to the light outlet/light outlet channel, while other optical filters are pulled away from the light outlet/light outlet channel and stored in the instrument. The optical filter 5 is fixed by the optical filter holder. Referring to FIGS. 10-11, there are the three-layer optical filter 5 as an example. FIG. 11(a) shows that the first optical filter is located in the light outlet/light channel for filtering the emitted light of the lamp 3, FIG. 11(b) shows that the first and second optical filters are located in parallel in the light outlet/light channel for filtering the emitted light of the lamp 3, FIG. 11(c) shows that the first and third optical filters are located in parallel in the light outlet/light channel for filtering the light emitted from the lamp 3. As an example, as shown in FIG. 11(a), after the lamp 3 is triggered, the emitted light passes through the first optical filter (with bands of 500-1200 nm), and the light after being filtered can directly shine the skin. As shown in FIG. 11(b), after the lamp 3 is triggered, the emitted light passes through the first optical filter (with bands of 500-1200 nm) and then passes through the second optical filter (with bands of 550-850 nm) to emit out, that is, the light photons acting on the skin have wave bands of 550-850 nm. In this embodiment, the lamp holder 35 can be provided with a simple structure. The lamp 3, the reflector 32, and the light source heat sink 33 are installed and fixed. The front end of the lamp holder forms a light outlet window. The opposite side walls of the light output channel are provided with slots 350 through the side walls. There are multiple slots 350 corresponding to the number of optical filters (each is fixed by the optical filter holder), and the shape of the slot is adapted for the filter. The plurality of slot 350 is arranged in parallel and is used for each optical filter to be pulled in or out of the light outlet/light outlet channel. The first optical filter may be fixedly set at the light outlet window to seal the lamp 3. In this embodiment, by operating a manual toggle mechanism provided on the beauty instrument housing for connecting each filter, or by operating the optical filter holder extending from the beauty instrument housing, combined with a moving guide track or slot accordingly, it can be conveniently manually switching different optical filters into/out of the light channel. Alternatively, an electric push rod or other electric push-pull mechanism of the prior art is provided inside the beauty instrument and is electrically connected to the control circuit board 90 to automatically control the push-pull movement of the optical filters for switching.

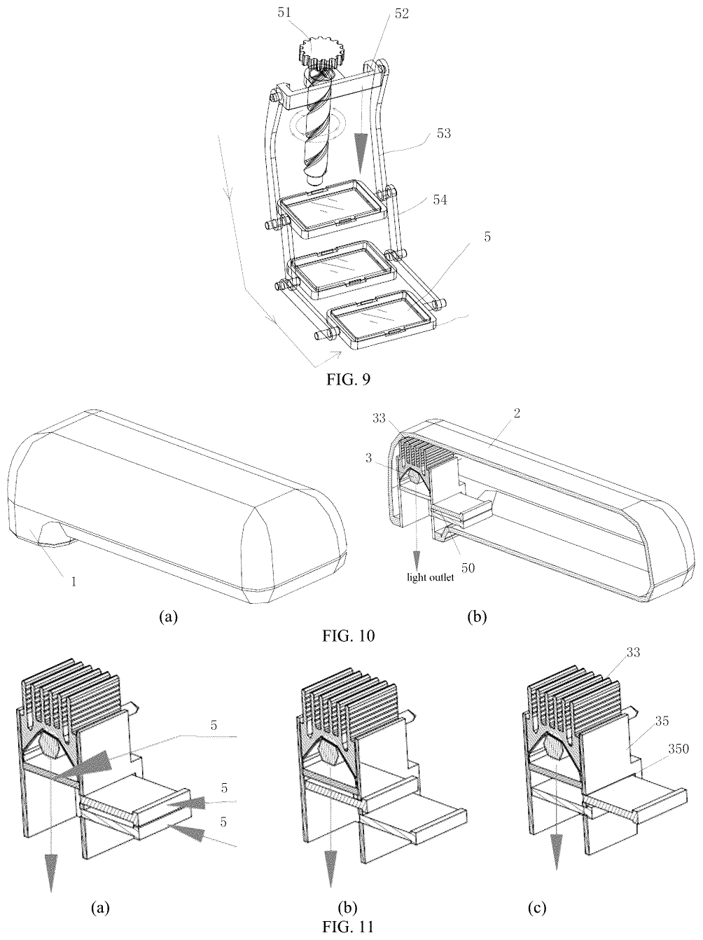

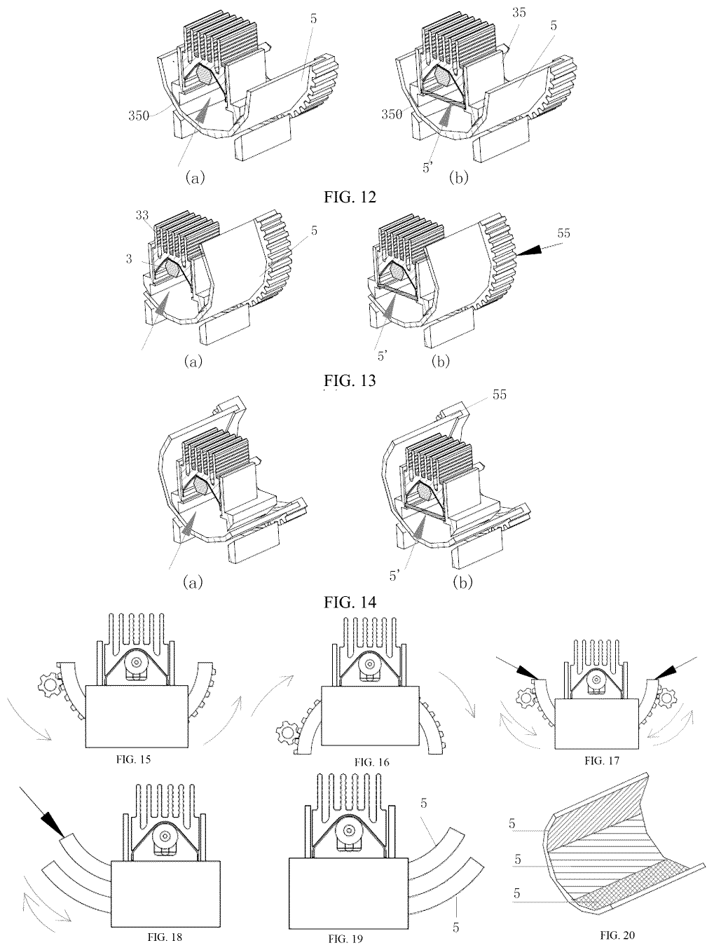

As shown in FIGS. 12-20, the optical filter assembly of the photon beauty instrument in a third embodiment, includes multiple optical filters 5 connected into an arc shape or formed on the same arc-shaped substrate, and the optical filters 5 are in an arc shape as a whole. Referring to FIG. 20, the optical filter arc can be formed by splicing three optical filters; or it can be coated in different areas of the same substrate to form different optical filter areas to serve as multiple optical filters. The optical filter arc in this embodiment takes up less space and can be used in various forms of beauty instruments. In this embodiment, a fixed optical filter 5′ and a movable and adjustable filter arc can be set in front of the lamp. Refer to FIGS. 12(b), 13(b), and 14(b), the fixed optical filter 5′ may be a plane optical filter, is fixed at the light outlet window at the front end of the lamp holder 35, covers an open of the reflector to seal the lamp 3 inside the reflector. Light wave emitted from the lamp 3 passes through the fixed optical filter 5′, and then is further filtered by the arc optical filter 5. In other embodiments, the fixed optical filter 5′ may not be provided, as shown in FIGS. 12(a), 13(a), and 14(a). In this embodiment, the slots 350 for the optical filters moving and switching are provided at the opposite side walls of the light outlet channel at the front end of the lamp holder 35, and the slot 350 is an arc-shaped slot that matches the arc-shaped optical filter 5. The arc optical filter 5 can be manually or automatically moved. As an embodiment, the arc optical filter holder 55 for fixing the arc optical filter 5 is provided with gear teeth; a motor-driven gear can be installed inside the body or the working head, and meshes with the gear teeth of the optical filter holder to drive the optical filter holder 55 to rotate, thereby moving the arc optical filter 5 and rotating the corresponding optical filter area to the light output channel for light filtering. Similarly, by manually turning a manual turning member connected to the optical filter holder 55, or by manually turning an outer edge of the optical filter holder 55 protruding from the beauty instrument housing, cooperating with a guide rail or guide slot, thereby, manually turn the optical filter holder 55 to move and switch different optical filters.

In other embodiments, a double-layer or multiple rows of slot 350 can be provided. FIGS. 18-19 illustrate the examples of two-layer slots 350. Multiple rows of optical filters are set in the same way as a single arc optical filter 5 (single-layer slot 350), thereby enabling the switching of multiple rows of optical filters.

It can be understood that the arc optical filter 5 can be arranged in an upward or downward direction, referring to FIGS. 15-16, other structures are the same or similar.

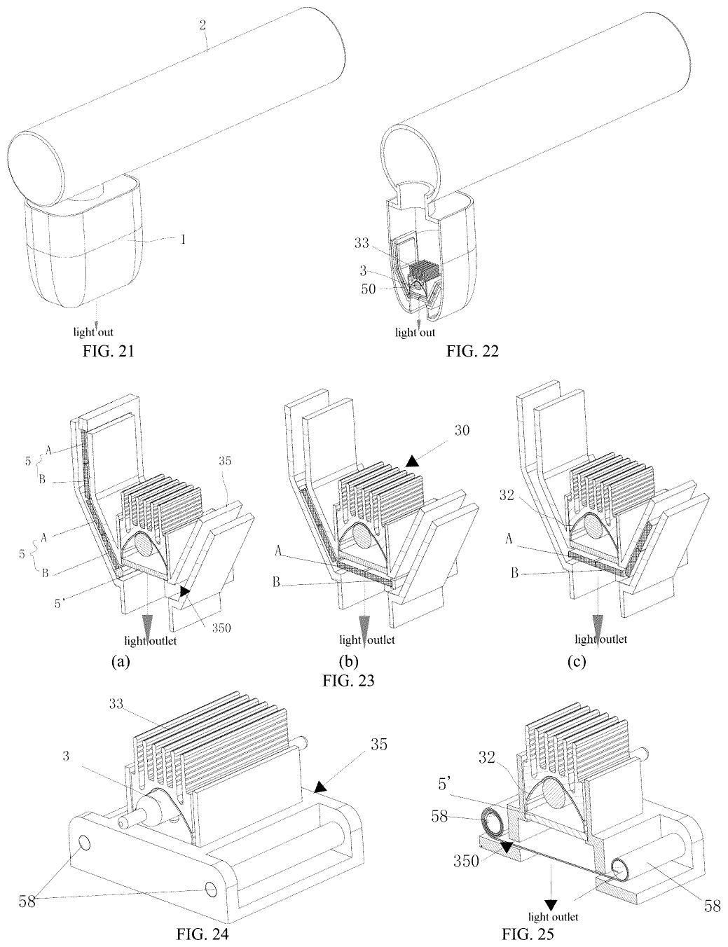

Referring to FIGS. 21-23, the optical filter assembly 50 of the photon beauty instrument in the fourth embodiment, includes multiple optical filters 5 in a form of a flexible connection, which can adapt to smaller corner spaces. The internal structure of the beauty instrument shown in FIG. 22 is simplified. In this embodiment, each optical filter 5 can be divided into two halves of sub-optical filters 5A and 5B. The A sub-piece and the B sub-piece are flexibly connected and moved to the light outlet together as one optical filter for filtering light. In this embodiment, the front end of the lamp holder 35 is installed with the lamp 3 and defines a light outlet channel/a light outlet (as shown by the arrows). A thermal conductive base on one side of the heat sink 33 has a surface consistent with a shape of the reflector 32 and is installed to fit together, and forms cooling fins on the other side. The front end of the lamp holder 35 has a double-layer wall. An interval between the double-layer walls forms a slot 350 for moving the optical filter 5. A cross-section of the double-layer wall is C-shaped, or similar to a C-shape, and the light source assembly 30 is installed inside the lamp holder. The light outlet window is provided with a fixed optical filter 5′ as the first optical filter. The second optical filter and the third optical filter are flexibly connected and accommodated in the slot 350, and can move along the slot. FIG. 23(a), FIG. 23(b) and FIG. 23(c) illustrate the switching states of the optical filter 5, that is, the first optical filter, the first optical filter plus the second optical filter, and the first optical filter plus the third optical filter respectively, is used for filtering the light waves emitted from the lamp 3.

In this embodiment, each optical filter 5 can be divided into two halves and spliced together by the flexible connection; and/or, for example, fixing the two halves by the optical filter holder 55 made of flexible plastic or elastomer material, or wrapping edges of the optical filter 5 using flexible plastic or elastomer material. Adjacent optical filter holders 55 are connected together through an integral connection part. Alternatively, adjacent optical filter holders 55 may be connected through soft or flexible material connectors, thereby forming a soft connection between multiple optical filters 5 or two parts of a single optical filter. The optical filter assembly in this embodiment can use the manual or electric adjustment mechanisms in the above-mentioned embodiments to move the optical filters for changing positions thereof.

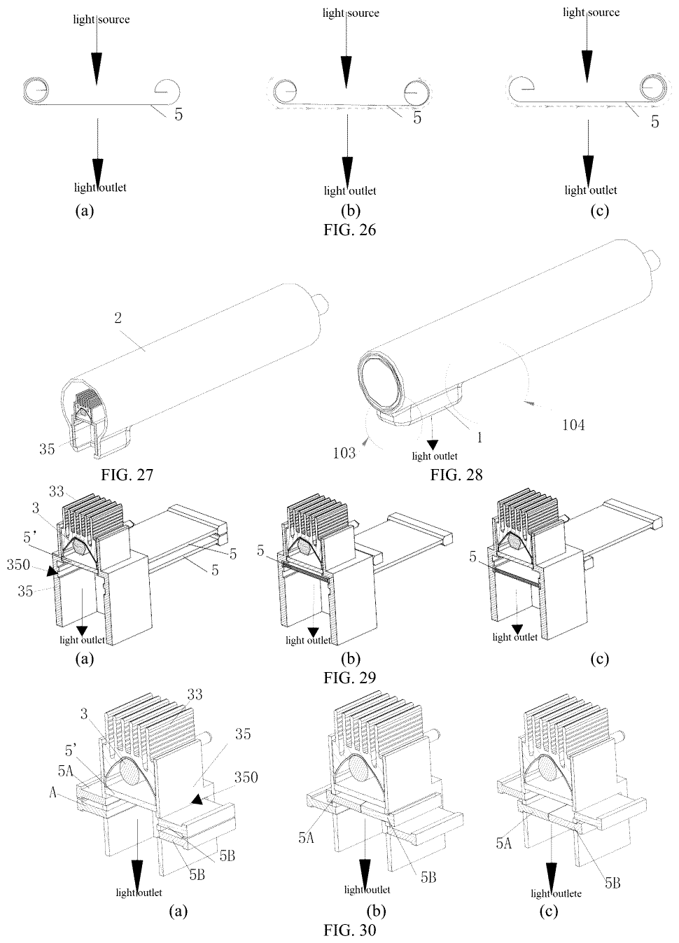

In the fifth embodiment of the photon beauty instrument shown in FIGS. 24-26, the optical filter assembly 50 adopts a soft film as optical filters, which takes up less space and can be adapted to various forms of instrument housings. The optical filter assembly 50 includes a roll of optical filter film. The optical filter film includes a plurality of optical filter areas along a conveying direction, and a plurality of optical filters 5 are formed correspondingly. The two ends of the optical filter film are respectively wound by a pair of rollers 58. The rotation of the roller 58 enables the corresponding optical filter area to be transferred to the light outlet. In this embodiment, the lamp holder 35 is provided with a rotating shaft seat located on both sides of the light outlet for rotatably installing the pair of rollers 58. The front end of the lamp holder 35 defines a light outlet channel and a light outlet/light outlet channel (as shown by the arrow in FIG. 25). The two opposite side walls of the light outlet channel or light outlet are provided with slots 350 respectively. The optical filter film has two ends wound on rollers 58 respectively and is disposed across the light outlet through the slots 350. The pair of rollers 58 are respectively rotatably installed outside the two side walls of the light outlet, and are arranged parallel to the slots 350. As shown in FIG. 25, the light outlet (or the light outlet window at the front end of the lamp holder) is provided with a fixed optical filter 5′ which is used as the first optical filter and covers on the light outlet (light outlet window). The optical filter film 5 is placed across the light outlet channel and located outside the fixed optical filter 5′. The pulsed light emitted from the lamp 3 is filtered by the fixed optical filter 5′ and the corresponding optical filter area (as the second optical filter) of the optical filter film, and then exits from the light outlet. Referring to FIG. 26, FIG. 26(a), FIG. 26(b) and FIG. 26(c), the switching states of positions of the optical filters 5 are illustrated, and the arrows indicate the emitting direction of the light from the light source. The optical filter film is located at the light outlet.

Referring to FIGS. 27-30, in the sixth embodiment of the photon beauty instrument, optical filters are set along a length direction of the working head, which is more suitable for a small space at the front end 103 of the instrument while a sufficient space at the rear end 104 (or there is insufficient space at one end/side and sufficient space at the other end/side) of the instrument. A plurality of optical filters 5 are moved into or out of the light outlet channel (light outlet) by pulling (translating). After the optical filters 5 are pulled back, they are stored in the working head housing, for example, in this embodiment, stored in the rear end of the working head. The two opposite side walls of the light outlet channel at the front end of the lamp holder 35 are respectively provided with slots 350. The optical filters 5 are slidably supported in a pair of slots in parallel and pushed forward into the light outlet channel at the front end. Multiple optical filters 5 are arranged in parallel and are slidably supported by slots 350 defined through the side walls of the light outlet channel. As shown in FIGS. 27 and 29, a fixed optical filter 5′ is provided in the light outlet/light channel as the first optical filter, covering the opening side of the light window or reflector, and multiple optical filters 5 are arranged in the light channel and located outside the fixed optical filter 5′. The pulse light from the lamp 3 is filtered by the fixed optical filter 5′ and the multiple optical filters 5 and then emitted from the light outlet. Referring to FIG. 29, FIG. 29(a), FIG. 29(b) and FIG. 29(c), position-switching states of the optical filters 5 are illustrates. FIG. 29(a) shows the fixed optical filter 5′ installed at the light outlet, while the second and three optical filters exit the light outlet and are stored in the rear end 104 of the working head. FIG. 29(b) and FIG. 29(c) respectively show that the second optical filter or the third optical filter is moved through the slot 350 into the light outlet/light outlet channel, and is spaced parallel to the fixed optical filter 5′. The pulse light from the lamp 3 is filtered by the fixed optical filter 5′ and the second or third optical filter 5 and then emitted from the light outlet, and then can directly act on the skin. The arrows in the figure indicate the light outlet channel/light outlet. The switching of the multiple optical filters 5 can be performed manually or electrically using the mechanism described in the above embodiment.

Referring to FIG. 30, the photon beauty instrument in the seventh embodiment has optical filters inserted and spliced from both sides, which take up a less space on one side, and is applicable for a smaller instrument. Specifically, slots 350 are respectively provided through the two opposite side walls of the light outlet channel at the front end of the lamp holder 35. The two halves of the optical filter 5A and 5B are slidably supported in a pair of slots, are pushed inward and translated into the light output channel and spliced into a whole piece of light optical filter. Multiple optical filters 5 are arranged in parallel and are slidably supported by multiple rows of slots 350 provided through the side walls of the light outlet channel. The light outlet window is provided with a fixed optical filter 5′ as the first optical filter which covers the opening side of the reflector to seal the lamp 3 in the reflector. Multiple optical filters 5 are provided in the light output channel outside the fixed optical filter 5′. The pulse light from the lamp 3 is filtered by the fixed optical filter 5′ and the multiple optical filters 5 and then emitted from the light outlet. FIG. 30(a), FIG. 30(b) and FIG. 30(c) show the position switching states of the optical filter 5. FIG. 30(a) shows the fixed optical filter 5′ located at the light outlet window, while the second and third optical filters each including half piece 5A and 5B exits the light output channel and to both sides respectively. FIG. 30(b) and FIG. 30(c) respectively show that the two halves of the second optical filter or the third optical filter are guided and slide through the slots 350, enter the light output channel and are spliced, and are arranged parallel to the fixed optical filter 5′. The pulse light from the lamp 3 is filtered by the fixed optical filter 5′ and the second or third optical filter 5 and then emitted from the light outlet, and can directly act on the skin. The arrows in the figures indicate the light outlet channel/light outlet. The switching of the multiple optical filters 5 can be performed manually or electrically by the mechanism described in the above embodiment.

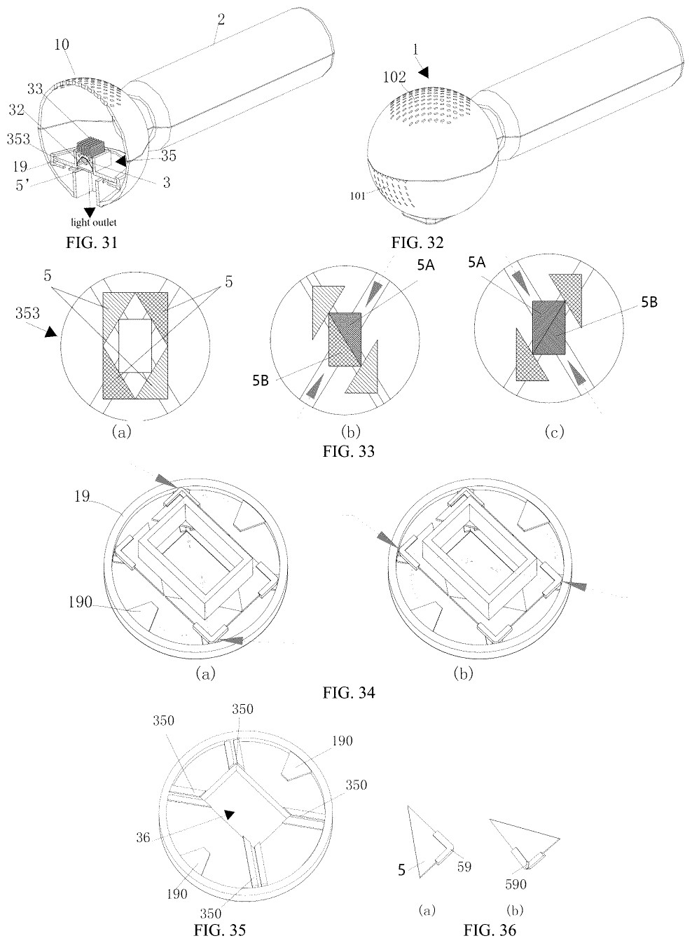

Referring to FIGS. 31-36, the photon beauty instrument in the eighth embodiment has the optical filters 5 spliced by diagonal rotation. It is suitable for the instruments that the working head 1 is round and has a relatively sufficient space. In this embodiment, the body 2 of the beauty instrument serves as a handheld and is shaped of a long cylinder; the working head 1 is spherical, and the front housing 10 is a spherical shell which is provided with air inlets 101, air outlets 102, and an opening as a light outlet. The lamp holder 35 is installed in the front housing 10. The lamp holder 35 is installed with a light source assembly in front end thereof, and includes a circular bottom plate 353. The lamp 3 is installed on one side of the bottom plate 353. The lamp 3 is enclosed by a reflector 32 and is installed on the bottom plate 353 of the lamp holder 35. The heat sink 33 is closely attached to the outside the reflector 32. One side of the thermal conductive base of the heat sink 33 has a surface consistent with the shape of the reflector 32, and is fitted closely with each other. Cooling fins are formed on the other side of the thermal conductive base to quickly conduct heat from the lamp to the heat sink for heat dissipation. A light outlet window 36 is formed at a center of the bottom plate 353 of the lamp holder, and a fixed optical filter 5′ (as the first optical filter) is installed and covers the opening side of the lamp reflector 32 to seal the lamp 3 inside the reflector 32. The lamp holder 35 forms a pair of side walls from the light outlet window 36 to the opening of the front housing 10 to define the light outlet channel (light outlet) therebetween. The other side of the bottom plate 353 of the lamp holder is movably installed with multiple optical filters 5 of the optical filter assembly. Specifically, two pairs (not limited to two pairs) of slots 350 as guide rails are formed through the bottom plate 353 radially from the central light outlet window 36. Preferably, each pair of slots 350 is located along a straight line, and the intersection point of the two straight lines corresponding to the two pairs of slots is located at the center of the bottom plate. The two optical filters 5 are slidably installed in two pairs of slots 350. Each optical filter 5 is divided into two halves, namely sub-optical filters 5A and 5B, which are slidably disposed in a pair of slots respectively and pushed toward the center to form a complete optical filter 5 located at the light outlet window 36. In this embodiment, the two optical filters 5 installed on the other side of the bottom plate 353 serve as the second and third optical filters respectively. When the sub-pieces 5A and 5B of one optical filter move closer to along the corresponding slots 350, the sub-pieces 5A and 5B of the other optical filter move away from each other. The optical filter 5 can be square, the sub-optical filters 5A and 5B are correspondingly triangular, and a splicing line is a diagonal of the square. A filer holder 59 is provided for fixing the edges of sub-optical filter 5A or 5B of the optical filter. A protruding bone 590 is provided on the filer holder 59. The protruding bone 590 is inserted into the slot 350 and slides without leaving the slot. The slot 350 and the protruding bone 590 are provided with snap edges corresponding to each other to prevent falling off. The filer holder 59 can be arranged in an L shape and fixed on a top corner of the sub-optical filter 5A or 5B of the optical filter to drive the sub-optical filter to slide.

The inner wall of the front housing 10 is correspondingly provided with an annular (or arc-shaped) slot 19. The periphery of the bottom plate 353 is inserted in the annular slot 19. The annular slot 19 supports the edges of the bottom plate 353 but is not fixed. A pair of protruding teeth 190 are provided on the edge of the annular (or arc-shaped) slot 19. The pair of protruding teeth 190 may be located on a straight line, protrude and extend toward the center of the bottom plate, and can be movably engaged with the filer holders 59 of a pair of sub-pieces 5A and 5B of each optical filter. In this embodiment, the straight lines of the two pairs of slots 350 and the pair of protruding teeth 190 are located along the diameters of the circular bottom plate 353. As an embodiment, the front housing 10 is divided into upper and lower hemispheres, which can be rotated relative to each other at a certain arc (or angle). The annular (or arc-shaped) slot 19 is provided along the inner wall of the lower hemispheric shell. Rotate the lower hemispheric shell to drive a pair of protruding teeth 190 rotating. When the protruding teeth 190 rotate to the slots of a pair of sub-pieces 5A and 5B of one optical filter, movably abut against the filer holders 59 of the pair of sub-pieces 5A and 5B of the optical filter, and push the pair of sub-optical filters 5A and 5B to move closer together until they are spliced; at the same time, the pair of sub-optical filters 5A and 5B push the other pair of sub-optical filters 5A and 5B of the other optical filter away from each other and slide to the edges of the bottom plate. Turn reverse the lower hemispherical shell, and the protruding teeth 190 can rotate to push the other pair of sub-pieces 5A and 5B of the other optical filter closer together until they are spliced. A protruding length of the protruding teeth 190 is set to promote the sub-pieces 5A and 5B splicing to a whole optical filter; the arc (or angle) of the forward and reverse rotation of the lower hemispherical shell is set to promote the sub-pieces 5A and 5B splicing to a whole optical filter respectively. In other embodiments, an adjusting ring 56 may be provided. The adjusting ring 56 may be rotated at a certain arc (or angle) between the upper and lower hemispheric shells of the front housing 10. The annular (or arc-shaped) slot 19 and a pair of protruding teeth 190 are disposed on the edge of the adjusting ring 56, and the edge of the bottom plate 353 is inserted in the annular (or arc-shaped) slot 19, and the protruding teeth 190 can movably push a pair of sub-pieces 5A and 5B of each optical filter closer together until they are spliced.

The pulsed light generated from the lamp 3 is emitted from the fixed optical filter 5′ at the light outlet window 36, and the emitted light after being further filtered by the second/third optical filter 5 in the light outlet channel can directly act on the skin.

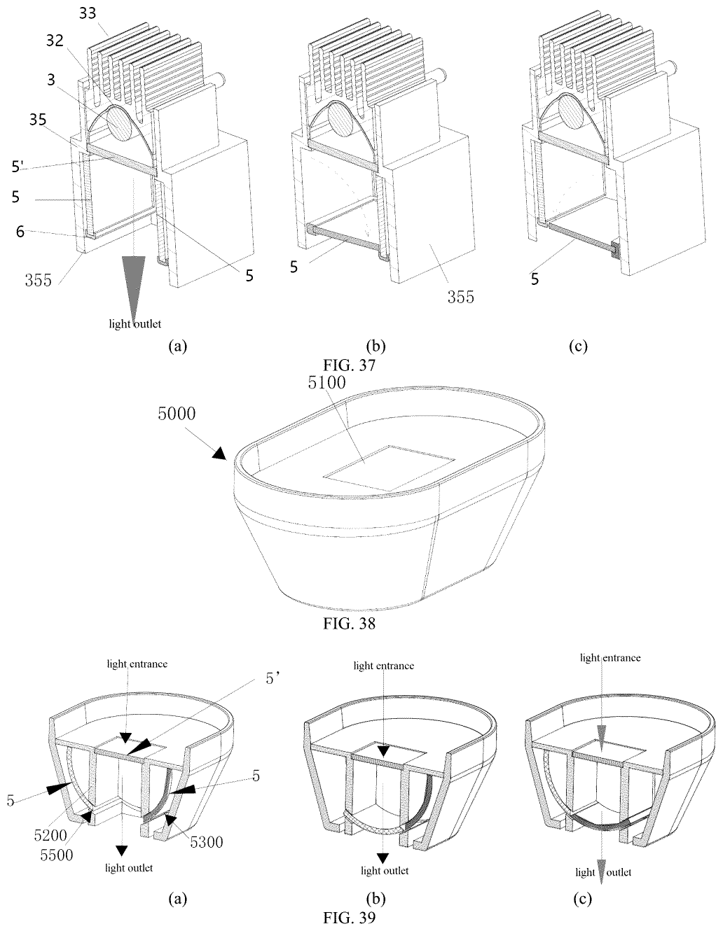

Referring to FIG. 37, the photon beauty instrument in the ninth embodiment has flippable optical filters, which take up a small space and can be applicable for all forms of beauty instruments, that is, it is suitable for various shapes and spaces of the beauty instrument. The lamp holder 35 is installed in the front housing 10 of the working head 1. The front end of the lamp holder 35 is installed with a light source assembly. The lamp 3 is enclosed by the reflector 32 and installed on the bottom plate 353 of the lamp holder 35. The heat sink 33 is closely attached to the outside the reflector 32. One side of the thermal conductive base of the heat sink 33 has a surface consistent with the shape of the reflector 32, and is fitted closely with each other. Cooling fins or sheets are formed on the other side of the thermal conductive base. A light outlet window is formed at the front end of the lamp holder, and a fixed optical filter 5′ (as the first optical filter) is installed at the light outlet window and covers the opening side of the lamp reflector 32 to seal the lamp 3 inside the reflector 32. The lamp holder 35 forms a pair of side walls 355 from the light outlet window to the opening of the front housing 10 to define the light outlet channel (light outlet) therebetween. as shown by the arrow in FIG. 37(a). The pair of side wall 355 is provided with rotation centers 6 formed by a pivot or a hinge for rotatably installing optical filters 5 respectively as the second optical filter and the third optical filter, the two optical filters 5 can respectively flip up around the rotation center 6 in the light outlet channel, for example, flip and switch between the vertical position and the horizontal position, and rotate to close the light outlet in the light outlet channel perpendicularly to the light emitting direction and parallel to the fixed optical filter 5′. FIG. 37(a) shows that the two optical filters (the second optical filter and the third optical filter) are located in the storage position, that is, close to the inner wall of the vertical side wall 355. FIG. 37(b) and FIG. 37(c) show the second optical filter or the third optical filter respectively rotated to a horizontal position to close the light outlet. The pulse light from the lamp 3 passes through the fixed optical filter 5′ and then is further filtered by the second optical filter or the third optical filter, and the emitted light can directly act on the skin. It can be understood that only one optical filter 5 can be provided, or multiple optical filters can be provided on the inner wall of the light outlet channel, and each level can be provided with one or two opposite optical filters 5. The photon beauty instrument in this embodiment can be a beauty instrument or hair removal instrument in the prior art, with the optical filter assembly installed in the light outlet channel outside the lamp. Or, the beauty instrument has a body (including appearance and internal structure) as the aforementioned embodiments

It can be understood that the rotation center 6 of this embodiment can be arranged at the top or the bottom, that is, the optical filters can be arranged in such a way that they can be flipped up or down. The optical filter is fixed by the optical filter holder, and the rotation center 6 may be arranged on the optical filter holder or engaged with the optical filter holder.

The photon beauty instrument in the above embodiments, a reflector mask and/or a thermoelectric cooling can be provided at the light outlet and protrude out of the opening of the front housing, so that the light outlet (light outlet channel) protrudes outward for a certain length, and the front end surface can be used for contact with skin. The thermoelectric cooling can use a transparent crystal as the cold side, which is directly used as a working surface in contact with the skin. Or a transparent crystal cooled by the cold side of the thermoelectric cooling attached thereto as the working surface, or an annular thermal conductive sheet is provided as the working surface to contact the skin.

FIGS. 38-47 show another embodiment of a photon beauty instrument with multi-optical filter, in such way that the optical filters are externally installed. Specifically, an accessory head 5000 is connected to the working head of the beauty instrument. The built-in optical filter assembly 50 in each of the above embodiments can be installed in the accessory head 5000 and connected to the working head of the photon beauty instrument. The accessory head is provided with a light outlet window 5100 for transmitting light into the accessory head, and a fixed optical filter 5′ can be disposed at the light outlet window 5100. The light outlet window 5100 of the accessory head is adapted to the light outlet of the working head 1 of the body of the beauty instrument. When the accessory head is connected to the working head, the light outlet window 5100 of the accessory head is just aligned with the light outlet of the working head 1. By operating the accessory head, switch the optical filter in the accessory head to the light outlet, so that the pulse light generated from the light source in the working head of the beauty instrument is filtered by the optical filter in the accessory head and then emitted for skin treatment. The arrows in FIGS. 38-47 show the light entrance and light exit of the accessory head. The accessory head is externally connected to the working head of the beauty instrument, which is equivalent to a detachable extension part or an external part of the working head. It can store one or more optical filters inside (a fixed optical filter can be set inside the working head), thereby filtering light waves from the light source, which has following advantages that: only one accessory head can install more than one optical filter for manual or electrical switching, which is convenient for storage, cleaning, low cost, and environmental protection.

It can be understood that the accessory head 5000 can be provided with a manual turning member connected to the optical filter assembly described in the above embodiments or the edge of the optical filter holder can protrude outside the housing to facilitate manual adjustment of the positions of the optical filters inside the accessory head. The electric adjustment mechanism in the above embodiments can also be provided on the accessory head. The accessory head is provided with a sub-circuit board and/or connector, and the body of the beauty instrument is provided with a corresponding connector. When the accessory head is attached to the working head, the circuit in the accessory head is electrically connected to the control circuit board 90 in the body of the photon beauty instrument, thereby the filter positions can be adjusted electrically.

FIG. 38 shows a structure of the accessory head 5000. A light outlet window 5100 is provided in the center of the top surface as a light entrance into the accessory head, and a pair of parallel side walls 5200 are provided inside. The interval between the parallel side walls forms a light outlet channel, that is, the light outlet. The space between the parallel side walls 5200 and the shell of the accessory head 5000 respectively forms a cavity 5500 for storing the optical filters 5 and for the optical filters 5 to enter and exit. A through slot 5300 is defined through each of the parallel side walls 5200 to connect the light output channel with the cavity 5500, allowing the optical filters 5 to slide into or out of the light outlet channel.

In the accessory head of the embodiment shown in FIG. 39, the optical filter 5 is arc-shaped, and a basic optical filter (the first optical filter) 5′ is fixed at the light outlet window, and arc optical filters as the second and third optical filters are installed at the left and right sides of the light output channel respectively. The arc-shaped optical filters 5 can move through the slot 5200 to the light outlet channel or to the storage cavity 5300, when move into the light outlet channel, it seals the light outlet channel laterally and filtering the incident light. After filtered, the light is emitted from the light outlet. FIGS. 39(a)-(c) show the switching states of the optical filters. FIG. 39(a) shows that only the fixed optical filter 5′ for filtering the incident light, and the second and third optical filters are stored in the cavity 5300 at both sides. FIG. 39(b) shows that the second optical filter slides into the light output channel, and the incident light is filtered by the fixed optical filter 5′ and the second optical filter 5 before being emitted; FIG. 39(c) shows that the third optical filter slides into the light output channel, and the incident light is filtered by the fixed optical filter 5′ and the third optical filter 5 before being emitted. It can be understood that multiple rows of optical filters can be provided at both sides of the light output channel. For example, multiple rows of optical filters at each side can be arranged in parallel and adjusted in combination. Multiple optical filters or multiple rows of optical filters can be switched to enter the light output channel for filtering light. The multiple arc-shaped optical filters in this embodiment are switched through rotational movement, and manual pulling/pull-out rods, connecting rods, etc. can be used for manual adjustment.

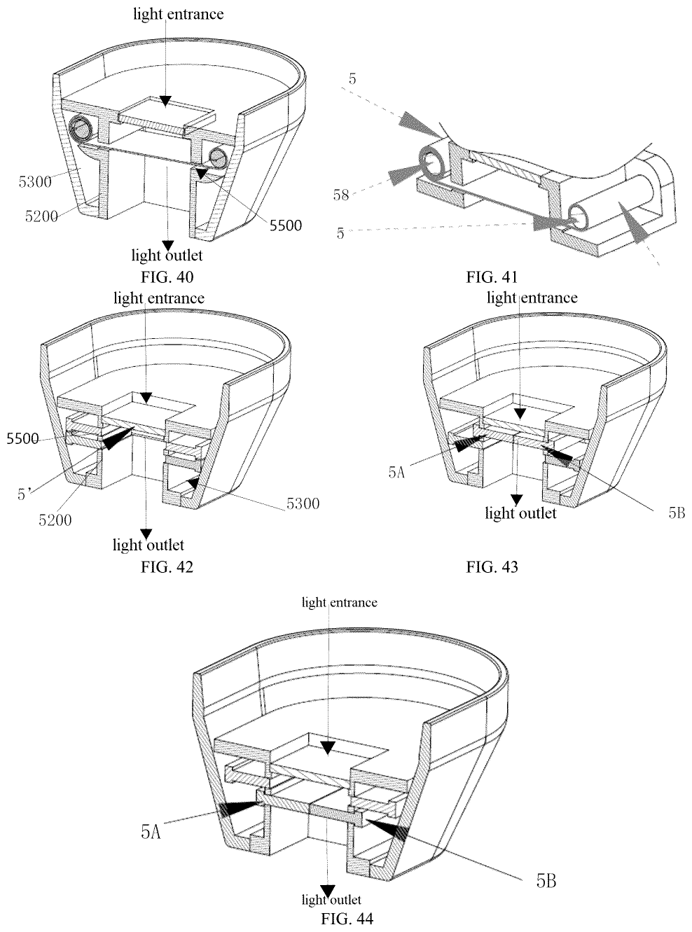

The accessory head in embodiment shown in FIGS. 40-41, includes the optical filter assembly using a soft film of optical filters, which takes up less space and can be adapted to various forms of housings. A roll of soft optical filter film is wound by a pair of rollers 58, and multiple parallel optical filter areas are provided along a transmission direction, which are respectively used as multiple optical filters 5. The rotation of the rollers 58 transports the soft optical filter film so that the selected filter area to the light output channel serves as the second optical filter. The pair of rollers 58 are accommodated in the storage cavities 5300 on both sides of the light outlet channel and are rotatably installed close to the side wall 5200. The soft optical filter film across both sides of the light outlet channel is transported from the through slot 5500 on the side wall to the left and right to transport different optical filter areas (optical filters) to the light outlet for filtering light. Referring to the foregoing embodiments, the roller 58 can be driven to rotate through a manual twisting way or an electrical way (motor and gear/motor and synchronous belt/motor and worm, etc.) to switch the optical filters.

In the accessory head of the embodiment shown in FIGS. 42-44, the optical filter assembly is in the form of spliced optical filters 5 inserted from both sides of the light outlet channel. The space requirement of one side is smaller, so it can adapt to smaller products. One or more rows of slot 5500 are provided through both side walls 5200 of the light outlet channel. One or more rows of optical filters 5 slide through the slots 5500 in the storage cavity 5300 on the left and right sides and the light outlet channel, so as to be stored in the cavity 5300 or enter the light outlet channel to cover the light outlet. In this embodiment, each optical filter 5 is divided into left and right sub-pieces 5A and 5B, which are simultaneously pushed into the light outlet channel and spliced into a whole optical filter located at the light outlet to filter the incident light. FIG. 42 shows that only the fixed optical filter 5′ filters the incident light, while the second and third optical filters are stored in the cavities 5300 on both sides. FIG. 43 shows the second optical filter (including the sub-pieces 5A and 5B on both sides) slides into the light output channel, and the incident light is filtered by the fixed optical filter 5′ and the second optical filter 5 before being emitted; FIG. 44 shows the third optical filter (including the sub-pieces 5A and 5B on both sides) slides into the light output channel, and the incident light is filtered by the fixed optical filter 5′ and the third optical filter 5 before being emitted.

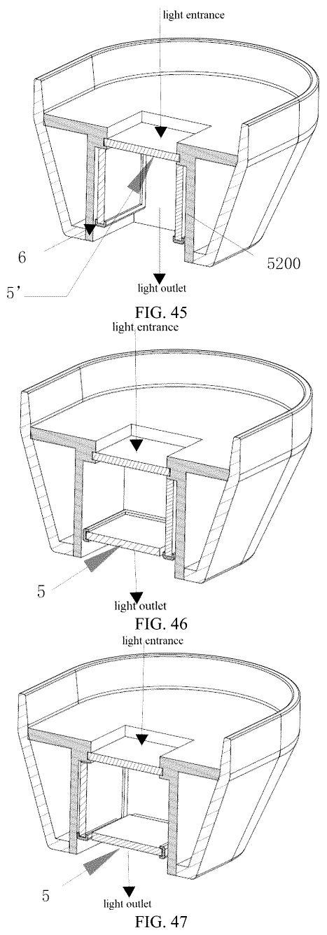

Referring to the accessory head of the photon beauty instrument shown in FIGS. 45-47, the optical filter assembly in this embodiment has flippable optical filters, which has a small space and can be adapted to all forms of beauty instruments and accessory heads, that is, it is suitable for various shapes and space design. The opposite side walls 5200 of the light output channel are each provided with a rotation center 6 formed by a pivot or a hinge, and optical filters 5 are installed respectively as the second optical filter and the third optical filter. The two optical filters 5 can be flipped around the rotation centers 6 in the light outlet channel respectively, for example, flip between a vertical position and a horizontal position, and rotate to close the light outlet in the light outlet channel perpendicular to the light outlet direction and parallel to the fixed optical filter 5′. FIG. 45 shows the two optical filters (the second optical filter and the third optical filter) located at the inner wall at a storage position. FIG. 46 and FIG. 47 show the second optical filter and the third optical filter rotated to the horizontal position respectively to close the light outlet. The pulsed light passes through the fixed optical filter 5′ and then is further filtered by the second optical filter or the third optical filter, and the emitted light can directly act on the skin. It can be understood that only one optical filter 5 can be provided, or multiple rows of optical filters can be flipped on the inner walls of the light outlet channel, and each row of optical filters can be provided with one or two opposite optical filters 5. It can be understood that the rotation center 6 in this embodiment can be disposed at the top or the bottom, that is, the optical filter can be flipped up or down. The optical filter is fixed by the optical filter holder, and the rotation center 6 is arranged on the optical filter holder or engaged with the optical filter holder.

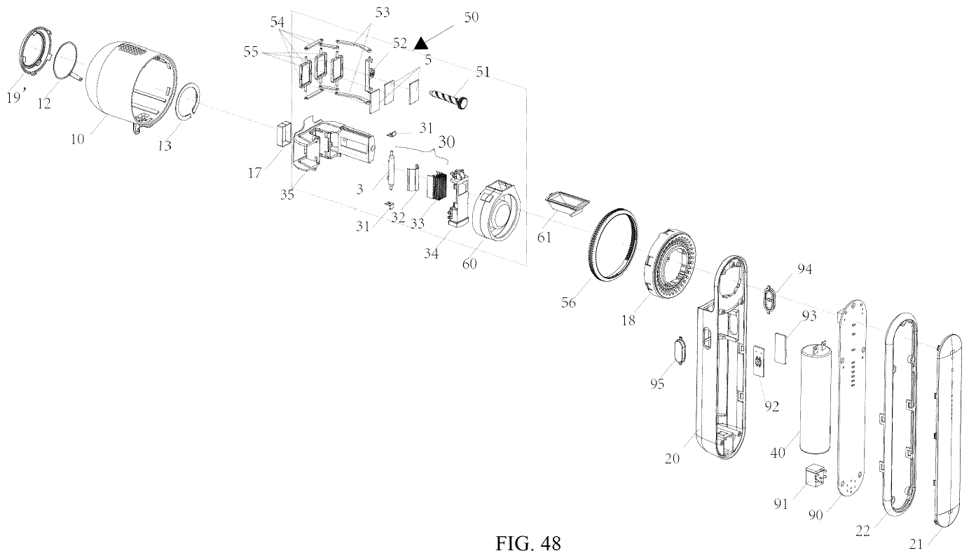

The photon beauty instrument in the tenth embodiment as shown in FIG. 48, compared with the photon beauty instrument shown in FIG. 1, the working head of the photon beauty instrument of this embodiment is not provided with the cooling function, that is, is not provided with the thermoelectric cooling and the heat sink 6, while is provided with a fan for air cooling and heat dissipation.

In the above embodiments, the optical filter assembly or the accessory head which are provided with built-in switchable optical filters, and are suitable for photonic beauty instruments with or without thermoelectric cooling.

In some embodiments, the wavelengths of the optical filter 5 (the light wave filtered by the optical filter) can be: 510 nm-1200 nm, 530 nm-1200 nm, 560 nm-1200 nm, 590 nm-1200 nm, 610 nm-1200 nm, 640 nm-1200 nm, or 645-750 nm.

For the optical filter assembly 5 used for the photon beauty instrument with hair removal function, the wavelength of its optical filters is preferably above 610 nm, for example, the optical filters with band of 610-1200 nm is used to transmit light with wavelengths greater than 610 nm to the working surface 10. In other embodiments, use at least one optical filter assembly 5 with a band of 645-750 nm, which is a dual-band filter that absorbs light with wavelengths below 645 nm and above 750 nm, and selectively transmits light with wavelength of 645-750 nm.

Examples of the wavelengths of the optical filter and the beauty effect of the photon beauty instrument include but are not limited to:
the optical filter with wavelengths of 430-1200 nm can be used to treat inflammatory acne;
the optical filter with wavelengths of 480-1200 nm can be used to treat acne and vascular lesions;
the optical filter with wavelengths of 530-1200 nm can be used to treat vascular (superficial small blood vessels) and pigmented lesions;
the optical filter with wavelengths of 560-1200 nm can be used to reduce wrinkles and treat pigmented and vascular lesions (deep and thick blood vessels);
the optical filter with wavelengths of 640-1200 nm can be used for hair removal, skin rejuvenation, and reducing facial redness; and
the optical filter with wavelengths of 690-1200 nm can be used for hair removal, reducing facial redness, etc.

The technical features of the above embodiments can be combined, changed or replaced to obtain different embodiments, and these embodiments all belong to the disclosure scope of the embodiments of the present invention. Some common structures or similar structures in the above embodiments are described in some embodiments but not described in other embodiments, while these common structures or similar structures are also applicable to those other embodiments, and all belong to the disclosure scope of the embodiments of the present application.

In the description of the present invention, it should be understood that the terms “length”, “width”, “upper”, “lower”, “front”, “rear”, “left”, “right”, “vertical”, “horizontal”, “top”, “bottom”, “inner”, “outside”, etc. are based on the orientations or positional relationships shown in the drawings, and are only for the convenience of describing the present invention, rather than indicating or implying that the device or component referred to must have a specific orientation or be constructed and operate in a specific orientation, and therefore cannot be considered as a limitation of the present invention.

In addition, the terms “first” and “second” etc. are only used for descriptive purposes and cannot be understood as indicating or implying relative importance or limiting the number of indicated technical features. Therefore, features defined as “first” and “second” etc. may include one or more of these features. In the description of the present invention, “plurality” means two or more than two, unless otherwise explicitly and specifically limited.

In the present invention, unless otherwise clearly stated and limited, terms such as “install”, “link”, “connect”, and “fix” should be understood in a broad meaning. For example, it can be a detachable fixed connection or an integration; it can be a mechanical connection, an electrical connection or a connection that can transmit data; it can be a direct connection or an indirect connection through an intermediate medium; it can be an internal connection between two components or between two components interaction relationship. For those of ordinary skill in the art, the specific meanings of the above terms in the present invention can be understood according to specific circumstances.

“A” or “an” mentioned in the specification or claims of the present invention includes one or more, except where it is clearly stated that there is only one. Similarly, “two” mentioned in the description or claims includes two or more, except where it is clearly stated that there are only two. Sometimes, the claims and description may include words such as “a plurality,” “one or more,” or “at least one,” however, in the absence of such a limitation, it does not mean it cannot be more than one.

Although embodiments of the invention have been shown and described, those of ordinary skill in the art will appreciate that various changes can be made to these embodiments without departing from the principles and spirit of the invention. Modifications, replacements and variations shall all fall within the scope of the present invention; the protection scope of the present invention is defined by the appended claims and their equivalent scope.

1. A photon beauty instrument, comprising: a housing, and a light source assembly, a power source unit and a control circuit board arranged in the housing; wherein the light source assembly, the power source unit and the control circuit board are electrically connected; a head of the photon beauty instrument defines a light outlet channel and a light outlet therein; light from the light source assembly is transmitted through the light outlet channel and emitted from the light outlet; the head of the photon beauty instrument is provided with an optical filter assembly with switchable optical filters therein; the optical filter assembly comprises one or more optical filters, or an optical filter film having multiple optical filter areas; and each optical filter or each optical filter area of the optical filter film is movable and switchable to enter the light outlet channel for filtering light transmitted through the light outlet channel.

2. The photon beauty instrument as claimed in claim 1, wherein multiple optical filters are connected in series by a connecting mechanism or spliced together to form linked optical filters; the linked optical filters are transported to the light outlet channel using a transmission mechanism or an adjustment mechanism for filtering light in the light outlet channel; or
one or more optical filters are each movably arranged in side walls of the light outlet channel, and are moved or rotated into the light outlet channel for filtering light transmitted through the light outlet channel.

3. The photon beauty instrument as claimed in claim 2, wherein a holder is provided in the head of the photon beauty instrument, and the holder defines the light outlet channel;
a side wall of the light outlet channel is provided with a slot or a rotating mechanism; the one or more optical filters or the optical filter film are moved into the light outlet channel through the slot for filtering light transmitted through the light outlet channel, or the one or more optical filters are rotated by the rotating mechanism into the light outlet channel for filtering light transmitted through the light outlet channel.

4. The photon beauty instrument as claimed in claim 3, wherein the slot is disposed in the holder for storing the optical filters or the optical filter film; the optical filters or the optical filter film are movable forward and backward and transported along the slot into the light outlet channel for filtering light.

5. The photon beauty instrument as claimed in claim 4, wherein the slot is arranged in the holder along a length direction of the holder and penetrate opposite sides of the holder; the optical filters are stored in the slot along the length direction and movable back and forth along the slot to the light outlet channel.

6. The photon beauty instrument as claimed in claim 3, wherein the optical filter is divided into two sub-optical filters, which are movable or rotatable to enter the light output channel and be spliced into one entire optical filter.

7. The photon beauty instrument as claimed in claim 3, wherein a storage cavity is formed outside the side wall of the light outlet channel, and is connected to the light outlet channel through the slot defined through the side wall of the light outlet channel; the optical filters or the optical filter film pass through the slot, are movable back and forth between the storage cavity and the light outlet channel, whereby being stored in the storage cavity or crossing the light outlet channel for filtering light.

8. The photon beauty instrument as claimed in claim 1, wherein the optical filters or optical filter film are arc-shaped or planar; positions of the optical filters or optical filter film in the optical filter assembly are manually switchable using a manual turning member or a lever or a connecting rod; or, positions of the optical filters or optical filter film are automatically adjustable by means of a motor driving a gear transmission, or a belt transmission, or a chain transmission, or a screw rod.

9. The photon beauty instrument as claimed in claim 3, wherein the optical filter assembly is arranged in the head of the photon beauty instrument, the holder is a lamp holder, the light source assembly is installed in one side of the lamp holder; the light source assembly includes a lamp and a reflector, and an opening side of the reflector form a light outlet window, a fixed optical filter covers the light outlet window to seal the opening side of the reflector; a heat sink is provided outside the reflector; a fan is installed in the head of the beauty instrument, and a housing of the head defines air inlets and air outlets, whereby air cooling and heat dissipation for the light source assembly is available.

10. The photon beauty instrument as claimed in claim 1, wherein the optical filter assembly is installed in an accessory head, the accessory head as an external component is connected to the head of the photon beauty instrument; a light outlet window is provided in a center of a top surface of the accessory head as a light entrance, which is connected with the light outlet of the head of the photon beauty instrument.

11. The photon beauty instrument as claimed in claim 10, wherein inside of the accessory head forms opposite side walls with a space therebetween forming a light outlet channel and a light outlet, and a space between the side walls and an outer shell of the accessory head forms a storage cavity for the optical filters or the optical filter film being stored therein or moving in or out; through slots are defined in the parallel side walls respectively, connects the light outlet channel of the accessory head to the storage cavity, whereby the optical filters or the optical filter film are slidable into or out of the light outlet channel.

12. The photon beauty instrument as claimed in claim 11, the optical filters are arc-shaped, a basic optical filter is fixed at the light outlet window of the accessory head, and the arc optical filters are installed at opposite sidewalls of the light output channel respectively; the arc-shaped optical filters are movable through the through slot to the light outlet channel or to the storage cavity.

13. The photon beauty instrument as claimed in claim 11, the optical filter assembly comprises a roll of soft optical filter film wound by a pair of rollers, and multiple parallel optical filter areas are provided along a transmission direction, which are respectively used as multiple optical filters; the pair of rollers are accommodated in the storage cavities on both sides of the light outlet channel, the soft optical filter film across both sides of the light outlet channel is transported from the through slot on the side wall forward and backward to transport different optical filter areas to the light outlet for filtering light.

14. The photon beauty instrument as claimed in claim 11, the optical filter assembly includes spliced optical filters inserted from opposite sidewalls of the light outlet channel; one or more rows of through slot are provided through opposite sidewalls of the light outlet channel, and one or more rows of optical filters are slidable through the through slots in the storage cavity and the light outlet channel, so as to be stored in the storage cavity or enter the light outlet channel; the optical filter is divided into sub-pieces, which are simultaneously pushed into the light outlet channel and spliced into a whole optical filter located at the light outlet to filter light.

15. The photon beauty instrument as claimed in claim 11, the optical filter assembly has flippable optical filters, opposite sidewalls of the light output channel are each provided with a rotation center for rotating install the optical filters, whereby the optical filters flippable around the rotation centers in the light outlet channel to close the light outlet for filtering light.
